(19) United States
(12) Patent Application Publication
(10) Pub. No.: US 2008/0103086 A1
(43) Pub. Date: May 1, 2008

(54) METHODS FOR THE TREATMENT OF DISEASES ASSOCIATED WITH THE SECRETION OF HMGB1

(76) Inventors: Jeon-Soo Shin, Seoul (KR); Ju Ho Youn, Seoul (KR)

Correspondence Address: JHK LAW, P.O. BOX 1078, LA CANADA, CA, 91012-1078, US

(21) Appl. No.: 11/552,945

(22) Filed: Oct. 25, 2006

(51) Int. Cl. G01N 33/68; A61K 31/00

(52) U.S. Cl. 514/1; 436/86

(57) ABSTRACT

The present invention relates to a pharmaceutical composition comprising an antagonist of the phosphorylation of HMGB1, and a method for treating a condition associated with activation of the inflammatory cytokine cascade comprising administering an effective amount of an said antagonist of HMGB1 phosphorylation.

### FIELD OF THE INVENTION

The present invention relates to methods for the treatment of diseases associated with the secretion of HMGB1.

### BACKGROUND OF THE INVENTION

The high mobility group box 1 (HMGB1) protein, a highly conserved, ubiquitous protein, was first purified almost 30 years ago as a nuclear protein. HMGB1 is involved in nucleosome stabilization and gene transcription (Lotze, M. T., and K. J. Tracey. Nat. Rev Immunol 5:331-342 (2005)), and it can also localize to the cell membrane of neurites for outgrowth and to the cell membranes of tumor cells for metastasis. HMGB1 is passively released by necrotic cells, though not by apoptotic cells, and triggers inflammation. HMGB1 also functions as a late mediator of endotoxemia, sepsis, and hemorrhagic shock in animals and human patients (Wang, H., O. et al, Science 285:248-251 (1999); Sunden-Cullberg, J. et al, Crit Care Med 33:564-573 (2005); Ombrellino, M., et al, Increased serum concentrations of high-mobility-group protein 1 in haemorrhagic shock. Lancet 354:1446-1447 (1999)). Specific inhibition of endogenous HMGB1 could reverse the lethality of established sepsis with HMGB1 antagonists (Yang, H. et al, Proc Natl Acad Sci USA 101:296-301 (2004)). HMGB1 is released from activated monocytes and macrophages and natural killer (NK) cells and behaves as a proinflammatory cytokine. Exposure to HMGB1 leads to various cellular responses, including the chemotactic cell movement of smooth muscle cells and monocytes and the release of proinflammatory cytokines, such as tumor necrosis factor (TNF)-α, interleukin (IL)-1, IL-6, and IL-8 (14). NK cells are in close, physical contact with immature dendritic cells (DCs). IL-18, produced by immature DCs, causes NK cells to produce HMGB1. HMGB1, in turn, causes dendritic cell maturation and Th1 polarization, events that initiate the adaptive immune responses.

HMGB1 contains two homologous DNA-binding motifs (HMG boxes A and B) and an acidic tail. It also contains two nuclear localization signals (NLSs) and two putative nuclear export signals (NESs), demonstrating that HMGB1 shuttles between the nucleus and cytoplasm through a tightly controlled mechanism.

WO 2004/044001 describes acetylated HMGB1 and its role as a mediator of the late phases of inflammation. However, the phosphorylation of HMGB1 has not been previously shown to have any regulatory role in the function of HMGB1 as a mediator of the inflammation. No evidence of phosphorylation, methylation, or glycosylation has previously been found in HMGB1 from calf thymus, mouse thymus, and activated human monocytes (Bonaldi, T. et al, Embo J 22:5551-5560 (2003)).

In this background, the present inventors surprisingly found that HMGB1 is phosphorylated in the animal cells and the translocation of the protein between the nucleus and the cytoplasm is regulated by the phosphorylation. The present invention thus provides a method of treating diseases associated with the secretion of HMGB1 protein.

### SUMMARY OF THE INVENTION

According to one aspect of the present invention there is provided pharmaceutical composition comprising an antagonist of the phosphorylation of HMGB1, and a pharmaceutically acceptable carrier, excipient or diluent The antagonist may regulate the phosphorylation process or it may affect the phosphorylated HMGB1, e.g. by removing the phosphorylation from the HMGB1 through phosphatase pathway. In one embodiment, the antagonist is the PKC inhibitors.

According to another aspect of the present invention there is provided a method for treating a condition in a subject wherein the condition is characterized by activation of an inflammatory cytokine cascade, comprising administering an effective amount of an antagonist of HMGB1 phosphorylation.

The present invention also provides a method of identifying an agent that regulates the phosphorylation of HMGB1, comprising the steps (a) determining the level of the phosphorylated HMGB1 in the presence and absence of said agent; (b) comparing the level of the protein determined in step (a); and (c) identifying said agent as a regulator by the differences in the phosphorylation of HMGB1 activity in the presence or absence of said compound. In one embodiment, the regulator is an antagonist of the HMGB1 phosphorylation. In another embodiment, the regulator is an agonist of the HMGB1 phosphorylation.

The present invention also provides a method for diagnosis and/or prognosis of the conditions associated with the activation of the inflammatory cascade comprising measuring the concentration of phosphorylated protein HMGB1 in a sample, and comparing that concentration to a standard for phosphorylated protein representative of a normal concentration range of phosphorylated HMGB 1 in a like sample, whereby higher levels of phosphorylated HMGB1 are indicative of the disease. In one embodiment the sample is a serum sample.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein.

FIGS. 1A-1D show that HMGB1 is phosphorylated by TNF-α or OA treatment in RAW 264.7 cells. (A) RAW 264.7 cells were treated with TNF-α (20 ng/mL for 16 h) or OA (100 nM for 8 h). The nuclear (Nu) and cytoplasmic (Cyt) proteins were separated and blotted with anti-HMGB1. (B) Metabolic [³²P] labeling of HMGB1 in RAW 264.7 cells. RAW 264.7 cells were metabolically labeled with [³²P]orthophosphate for 4 h, and stimulated with OA (100 nM), TNF-α (20 ng/mL) and LPS (100 ng/mL) for 2 or 8 h. WCLs were immunoprecipitated with rabbit anti-HMGB1 from two different vendors of BD PharMingen (BD) and Upstate Biotechnology (UP). The proteins were resolved and transferred to nitrocellulose membrane and visualized by autoradiography. (C) RAW 264.7 cells were treated with TNF-α, and WCLs were immunoprecipitated with anti-pSer, anti-pTyr, and anti-pThr and blotted with anti-HMGB1. WCL was loaded as an HMGB1 control (lane 1). Anti-pAKT was used for a control antibody (lane 3). (D) RAW 264.7 cells were treated with TNF-α for the indicated time. WCLs were immunoprecipitated with anti-HMGB1, blotted with anti-pSer and reblotted with anti-HMGB1. The same culture supernatants were concentrated, separated, and blotted with anti-HMGB1.

FIGS. 2A-2D show that effects of HMGB1 phosphorylation on its location in RAW 264.7 cells and human PBMo cells. RAW 264.7 cells (A) and human PBMo cells (B) were treated with OA (100 nM for 8 h), and immunofluorescent staining was performed to observe the HMGB1. TNF-α (20 ng/mL for 16 h) was used as a positive control cytokine. HMGB1 was exclusively observed in the nuclei of the unstimulated (medium) RAW 264.7 and PBMo cells, but moved to the cytoplasm after OA treatment. (C) Western blot analysis of HMGB1 protein in the culture supernatants of PBMo cells, which were from (B). (D) HMGB1 in the nucleus is transported to the cytoplasm by phosphorylation. RAW 264.7 cells were transfected with a wild type HMGB1-GFP plasmid and cultured for 24 h. And then the cells were treated with 2 μg/mL CHX for 1 h followed by OA treatment for 4 h or by TSA treatment for 2 h and green fluorescent images were observed. Bar: 10 μm.

FIGS. 3A-3B show nuclear import assay of HMGB1. (A) Western blot analysis of His-tagged HMGB1-GFP, GST-GFP and GFP proteins. Six-His-tagged HMGB1-GFP protein was expressed in E. coli BL21 (DE3) pLysE for a nuclear import assay. These proteins were purified using a Ni²⁺-NTA column and blotted with anti-GFP. Each protein was observed at the predicted size. (B) Nuclear import assay of HMGB1. HeLa cells were permeabilized with digitonin and incubated for 1 h at 22° C. with the complete transport mixture. The transport mixture contained recombinant import protein and HeLa cell-derived cytosol, which was preincubated with an ATP-regenerating system in the presence or absence of OA. The cells were fixed and immediately observed by fluorescent microscopy. Bar: 10 μm.

FIGS. 4A-4C show the binding of HMGB1 to nuclear import proteins. (A) GST-KAP-α1, -2, -3, -4, -5, -6, and -β1 fusion proteins immobilized on glutathione-Sepharose 4B beads were incubated with WCLs of RAW 264.7 cells overnight at 4° C. Sepharose-bound proteins were separated and the membrane was blotted with anti-HMGB1 and reblotted with anti-GST. WCL was loaded as an HMGB1 control (lane 1), and GST protein was used as a negative control (lane 2). (B) Six His-tagged wild type HMGB1 and boxes A (aa 1-87) and B (aa 88-162) HMGB1 proteins were purified from E. coli BL21 and identified at expected size by Coomassie blue staining. HMGB1 and GST-KAP-α1 was incubated for 2 h at 4° C., and the precipitate was blotted with anti-His for HMGB1 and reblotted with anti-GST for KAP-α1. (C) GST-KAP-α1, immobilized on glutathione-Sepharose beads, was incubated with WCLs of RAW 264.7 cells which were treated with OA, TSA, or TNF-α. The precipitates were blotted with anti-HMGB1 and reblotted with anti-GST.

FIG. 5A-5D show the effect of phosphorylation of HMGB1 on binding to KAP-α1. (A) Schematic presentation of mutated HMGB1-GFPs. Serine residues were point-mutated into alanine (A) or glutamic acid (E). The first green box (aa 28-53) is NLS1 (dot box) and the adjacent serine-containing region, and the second green box (aa 179-185) is NLS2. Boxes A and B, the acidic tail, and the amino acid numbers are marked. WT: wild-type. (B) RAW 264.7 cells were co-transfected with Flag-tagged KAP-α1 and each HMGB1-GFP mutant plasmid. After 24 h, WCLs were prepared, immunoprecipitated with anti-GFP, and subjected to Western blotting. The membranes were blotted with anti-FLAG and reblotted with anti-GFP. FLAG-KAP-α1 levels were observed to determine whether equal amounts of WCLs were loaded. The reciprocal experiments were also performed (C). The molecular weight of HMGB1-GFP is similar to that of the Ig heavy chain, and the bands are located just below the Ig heavy chain bands.

FIGS. 6A-6E show Mutation of HMGB1 NLS sites alters subcellular distribution of HMGB1. (A) RAW 264.7 cells were transfected with wild-type and each mutant HMGB1-GFP plasmid and immunofluorescent assays were performed 24 h later without any treatment. (B, C, and D) OA (100 nM for 4 h), TSA (10 ng/mL for 2 h), and TNF-α (20 ng/mL for 16 h) were applied 24 h after transfection to observe the effect on HMGB1 nuclear export by phosphorylation, acetylation, or both. Some cells showed no GFP, implying no transfection. (E) Secretion of wild-type and each mutant HMGB1-GFP was tested 24 h after transfection by Western blotting. The culture supernatants were concentrated and blotted with anti-GFP. Bar: 10 μm.

FIG. 7 shows In vitro protein kinase assay of HMGB1. Six His-tagged wild type (WT) HMGB1 and HMGB1 NLS1/2A, both of which were not tagged with GFP, were purified from E. coli. Two μg of each protein was incubated with 50 ng of PKC, 500 U of casein kinase 11 and 20 U of cdc2 kinase in the presence of 5 μCi [γ-³²P]ATP for 40 min at 30° C. Each sample was added with 5× sample buffer to stop the reaction and separated with 12% SDS-PAGE. Autoradiography was performed after drying.

FIG. 8 shows Inhibition of HMGB1 secretion by Protein Kinase C inhibitors. RAW 264.7 cells were treated with 200 ng/mL of LPS alone or combined with Gö 6983 (PKC inhibitor) at the indicated concentrations (μM) for 16 h. SB203580 (p38 inhibitor), PD098059 (ERK1/2 inhibitor), Bay 11-7082 (NF-κB inhibitor) were used for comparison. The supernatants were harvested and separated with 12% SDS-PAGE and Western blot was performed to observe the level of HMGB1.

### DETAILED DESCRIPTION OF THE INVENTION

The term “regulate” as used herein refers to a change or alteration in the phosphorylation of HMGB1. The “regulation” therefore includes inhibition of phosphorylation, e.g. by compounds which block the protein kinases. The “regulation” also includes the activation of the HMGB1 phosphorylation, e.g. by compounds which stimulate the activity of protein kinases or by compounds which inhibit protein phosphatase pathway. The “regulator” of the present invention could be in the form of a chemical compound, or an mixture (for example, extract made from biological materials), proteins, carbohydrates, small molecules, or nucleic acids. Regulators are evaluated for their activity as antagonist or agonist of the HMGB1 phosphorylation. Such regulators can be screened using the methods described herein.

The term “antagonist”, is intended to refer broadly to any agent which lowers the level of phosphorylation of HMGB1 protein, and it is used interchangeably with “inhibitor”. “Agonist” is used to refer to any agent which increases the level of phosphorylation of HMGB1 protein, and it is used interchangeably with “activator”

### Pharmaceutical Composition and Method of Treatment

In one embodiment the present invention provides a pharmaceutical composition and method for treating diseases characterized by activation of an inflammatory cytokine cascade, particularly sepsis, including septic shock and ARDS (acute respiratory distress syndrome), comprising administering an effective amount of an antagonist to HMGB1 phosphorylation.

A pharmaceutical composition of the present invention comprises an antagonist of the phosphorylation of HMGB1, and a pharmaceutically acceptable carrier, excipient or diluent. The antagonist may regulate the phosphorylation process or it may affect the phosphorylated HMGB1, e.g. by removing the phosphorylation from the HMGB1 through phosphatase pathway. In one embodiment, the antagonist is the PKC inhibitors. In another embodiment, the antagonist is the activator or protein phosphatases.

### Phosphorylation of HMGB1

HMGB1 is a member of the B family of HMG proteins. cDNAs coding for HMGB1 have been cloned from human, rat, mouse, mole rat, trout, hamster, pig and calf cells, and is believed to be abundant in all vertebrate cell nuclei. The protein is highly conserved.

The inventors discovered that in the cells, the HMGB1 is phosphorylated at Ser residue and the phosphorylated HMGB1 was relocated to the cytoplasm. In most cells HMGB1 shuttles continually from the nucleus to the cytoplasm, but the equilibrium is almost completely shifted towards a nuclear accumulation. Treatment of cells with phosphatase inhibitor (OA) caused phosphorylation of the protein HMGB1 (Example 2). The phosphorylation also blocked re-entry of the protein to nucleus (Example 4), which suggests that the phosphorylation plays an important role in the controlled shuttling mechanism of the HMGB1. The inventors also showed that Protein Kinase C phosphorylates HMGB1 (Example 8 and Example 9).

HMGB1 can be phosphorylated at multiple sites. HMGB 1 contains two independent NLSs, and the phosphorylation of both NLS regions of HMGB1 was required for its relocation to the cytoplasm. Example 5 shows that HMGB1 binds to KAP-α1, a shuttling receptor protein, and the binding was affected by the phosphorylation of the HMGB1.

Phosphatase inhibitor (for example, OA) caused hyperphosphorylation of HMGB1 and the relocation of the protein to the cytoplasm. It was demonstrated that Protein Kinase C but neither Casein Kinase II nor cdc2 phosphorylated HMGB1 (Example 8). When the cells were treated with Protein Kinase C inhibitor, Go6983 (2-[1-(3-dimethylaminopropyl)-5-methoxyindol-3-yl]-3-(1H-indol-3-yl) Maleimide), the secretion of HMGB1 was inhibited.

### Therapeutic Composition

Acceptable carriers or diluents for therapeutic use are well known in the pharmaceutical art. Where appropriate, the pharmaceutical compositions can be administered by any one or more of administration route known in the art.

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (eg using slow release molecules by the intraperitoneal route or by using cells e.g. monocytes or dendrite cells sensitised in vitro and adoptively transferred to the recipient). Depending on the route of administration, the peptide may be required to be coated in a material to protect it from the action of enzymes, acids and other natural conditions which may inactivate said ingredients.

For example, the low lipophilicity of the peptides will allow them to be destroyed in the gastrointestinal tract by enzymes capable of cleaving peptide bonds and in the stomach by acid hydrolysis. In order to administer peptides by other than parenteral administration, they will be coated by, or administered with, a material to prevent its inactivation. For example, peptides may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the peptides are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

### Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, thus requiring only a fraction of the systemic dose.

### Method of Treatment

According to another aspect of the present invention there is provided a method for treating a condition in a subject wherein the condition is characterized by activation of an inflammatory cytokine cascade, comprising administering an effective amount of an antagonist of HMGB1 phosphorylation.

Diseases and conditions mediated by the inflammatory cytokine cascade are numerous. An inflammatory condition that is suitable for the methods of treatment described herein can be one in which the inflammatory cytokine cascade is activated. In one embodiment, the inflammatory cytokine cascade causes a systemic reaction, such as with endotoxic shock. In another embodiment, the inflammatory condition is mediated by a localized inflammatory cytokine cascade, as in rheumatoid arthritis. Nonlimiting examples of inflammatory conditions that can be usefully treated using the antibodies and antigen-binding fragments of the present invention include, e.g., diseases involving the gastrointestinal tract and associated tissues (such as ileus, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, coeliac disease, hepatitis, Crohn's disease, enteritis, and Whipple's disease); systemic or local inflammatory diseases and conditions (such as asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, and sarcoidosis); diseases involving the urogenital system and associated tissues (such as septic abortion, epididymitis, vaginitis, prostatitis, and urethritis); diseases involving the respiratory system and associated tissues (such as bronchitis, emphysema, rhinitis, cystic fibrosis, pneumonitis, adult respiratory distress syndrome, pneumoultramicroscopic silicovolcanoconiosis, alveolitis, bronchiolitis, pharyngitis, pleurisy, and sinusitis); diseases arising from infection by various viruses (such as influenza, respiratory syncytial virus, HIV, hepatitis B virus, hepatitis C virus and herpes), bacteria (such as disseminated bacteremia, Dengue fever), fungi (such as candidiasis) and protozoal and multicellular parasites (such as malaria, filariasis, amebiasis, and hydatid cysts); dermatological diseases and conditions of the skin (such as burns, dermatitis, dermatomyositis, sunburn, urticaria warts, and wheals); diseases involving the cardiovascular system and associated tissues (such as stenosis, restenosis, vasculitis, angiitis, endocarditis, arteritis, atherosclerosis, thrombophlebitis, pericarditis, congestive heart failure, myocarditis, myocardial ischemia, periarteritis nodosa, and rheumatic fever); diseases involving the central or peripheral nervous system and associated tissues (such as Alzheimer's disease, meningitis, encephalitis, multiple sclerosis, cerebral infarction, cerebral embolism, Guillame-Barre syndrome, neuritis, neuralgia, spinal cord injury, paralysis, and uveitis); diseases of the bones, joints, muscles and connective tissues (such as the various arthritis and arthralgias, osteomyelitis, fasciitis, Paget's disease, gout, periodontal disease, rheumatoid arthritis, and synovitis); other autoimmune and inflammatory disorders (such as myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, and Reiter's syndrome); as well as various cancers, tumors and proliferative disorders (such as Hodgkins disease); and, in any case the inflammatory or immune host response to any primary disease.

In one embodiment, the condition is selected from the group consisting of sepsis, allograft rejection, arthritis (e.g., rheumatoid arthritis), asthma, atherosclerosis, restenosis, lupus, adult respiratory distress syndrome, chronic obstructive pulmonary disease, psoriasis, pancreatitis, peritonitis, burns, myocardial ischemia, organic ischemia, reperfusion ischemia, Behcet's disease, graft versus host disease, Crohn's disease, ulcerative colitis, ileus, multiple sclerosis, and cachexia. In another embodiment, the condition is selected from the group consisting of sepsis, arthritis (e.g., rheumatoid arthritis), asthma, lupus, psoriasis, inflammatory bowel disease and Crohn's disease.

As used herein, an “effective amount” or “therapeutically effective amount” is an amount sufficient to prevent or decrease an inflammatory response, and/or to ameliorate and/or decrease the longevity of symptoms associated with an inflammatory response. The amount of the composition of the invention can be determined, for example, by administering the composition to an animal models. In addition, in vitro assays may optionally be employed to help identify optimal dosage ranges.

Selection of the preferred effective dose can be determined (e.g., via clinical trials) by a skilled artisan based upon the consideration of several factors that are known to one of ordinary skill in the art. Such factors include, e.g., the condition or conditions to be treated, the severity of the subject's symptoms, the subject's age, the subject's body mass, the subject's immune status, the response of the individual subject, and other factors known by the skilled artisan to reflect the accuracy of administered pharmaceutical compositions.

### Method for Diagnosis and Prognosis

The present invention also provides a method for diagnosis and or prognosis of the conditions associated with the activation of the inflammatory cascade comprising measuring the concentration of phosphorylated protein HMGB1 in a sample, and comparing that concentration to a standard for phosphorylated protein representative of a normal concentration range of phosphorylated HMGB 1 in a like sample, whereby higher levels of phosphorylated HMGB1 are indicative of the disease. In one embodiment the sample is a serum sample. The diagnostic method may also be applied to other tissue or fluid compartments such as cerebrospinal fluid or urine. The diagnostic assay may use anti-phosphorylated HMGB1 antibodies. Alternatively, the antibodies specific to HMGB1 and anti-p-amino acid antibody, such as anti-pSer antibody, could be used in combination simultaneously or sequentially. The diagnostic procedure can utilize standard antibody-based techniques such as ELISA assays and Western blot techniques.

### Method for Identifying Regulators

The present invention also provides a method of identifying an agent that regulates the phosphorylation of HMGB1, comprising the steps (a) determining the level of the phosphorylated HMGB1 in the presence and absence of said agent; (b) comparing the level of the protein determined in step (a); and (c) identifying said agent as a regulator by the differences in the phosphorylation of HMGB1 activity in the presence or absence of said compound. In one embodiment, the regulator is an antagonist of the HMGB1 phosphorylation. In another embodiment, the regulator is an agonist of the HMGB1 phosphorylation. To determine the level of phosphorylation, antibodies that recognize the phosphorylated HMGB1 could be used. Or, the phosphorylation could be monitored by radiolabeling method, for example, using [³²P] orthophosphate. Antibody techniques and radiolabeling techniques are all known in the art.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

### Example 1.1 Cell Culture

Murine macrophage RAW 264.7 cells (American Type Culture Collection, Manassas, Va.) and HeLa cells were cultured at 37° C. under 5% CO₂ in Dulbecco's modified Eagle's medium (DMEM) supplemented with 10% FBS (In vitrogen Life Technologies), 100 U/mL penicillin, 100 μg/mL streptomycin, and 2 mM L-glutamine. Human peripheral blood monocytes (PBMO) were harvested from the adhesive cells on the culture flask, after yielding peripheral blood mononuclear cells, by Ficoll-hypaque gradient centrifugation. Human recombinant TNF-α (R&D Systems), OA (Calbiochem), trichostatin A (TSA, Sigma-Aldrich), and cycloheximide (Sigma) were purchased.

### Example 1.2 Western Blotting Analysis

To analyze the secretion of HMGB1 in the supernatants, culture media were replaced with serum-free OPTI-MEM (Gibco BRL) medium and concentrated with Amicon Centricon filtration (Millipore) after removing cell debris, and Western blot analysis was performed. The cytoplasmic and nuclear fractions from 5×10⁶ cells were separated using a digitonin-based method (Bird, C. H. et al, Mol Cell Biol 21:5396-5407 (2001)) to observe the levels of HMGB1 in each fraction. The cells were lysed using 1% Nonidet P-40 buffer containing a protease inhibitor cocktail (Sigma), and the protein concentrations were measured by Bradford assay (Biorad) for the analysis of whole cell lysates (WCLs). The protein samples underwent 12% SDS-PAGE and were transferred to a nitrocellulose membrane. Western blot analysis was performed using rabbit anti-HMGB1 (BD Pharmingen) and HRP-labeled goat anti-rabbit Ig as primary and secondary antibodies, respectively. The signals were revealed with enhanced chemiluminescence (ECL, Pierce).

### Example 1.3 Immunoprecipitation

To identify the phosphorylated residues of the HMGB1 protein, TNF-α-treated RAW 264.7 cells were lysed with a protease inhibitor cocktail. Cell homogenates were centrifuged at 20,000 g for 15 min and precleared by incubation with protein G-Sepharose (Amersham) at 4° C. for 30 min. The precleared extracts (500 μg) were incubated with rabbit polyclonal anti-pSer, anti-pTyr and anti-pThr (all from Chemicon) and then protein G-Sepharose was added and incubated for 3 h at 4° C. Immune complexes were collected by centrifugation and washed with lysis buffer. Collected complexes were fractionated by SDS-PAGE, transferred to membranes and blotted with anti-HMGB1 for detection. Anti-pAKT (Cell signaling) was used as a negative control. To investigate the time-dependent phosphorylation of HMGB1, the WCLs of RAW 264.7 cells treated with TNF-α for the indicated time were immunoprecipitated with anti-HMGB1 and subjected to Western blot analysis using anti-pSer.

### Example 1.4 Immunofluorescence and GFP Imaging

Cells were cultured in LabTek II chambers (Nalgene) and were fixed in 3.7% paraformaldehyde in PHEM buffer (60 mM PIPES, 25 mM HEPES, 10 mM EGTA, 4 mM MgSO4, pH 7.0) for 10 min at RT. After fixation, the cells were washed with PBS and incubated for 3 min at 4° C. with HEPES-based permeabilization buffer containing 300 mM sucrose and 0.2% Triton X-100. The cells were blocked with 0.2% BSA in PBS for 15 min and were incubated with rabbit anti-HMGB1 for 1 h at RT. After 3 washes with blocking solution, secondary antibody FITC-conjugated goat anti-rabbit Ig (BD Pharmingen) was added. Cells expressing various HMGB1-EGFP proteins were stained with DAPI and the cells were observed with a BX51 fluorescent microscope (Olympus). Cells expressing HMGB1-GFP and its derivatives were fixed as described above.

### Example 1.5 Nuclear Import Assay in Digitonin-Permeabilized Cells

Nuclear import assays were performed with minor modification as previously described (Adam, S. A., R. S. Marr, and L. Gerace. 1990. J Cell Biol 111:807-816 (1990). Briefly, HeLa cell cytosol was first prepared. For this, HeLa cells at a density of 5×105 cells/mL were harvested and washed twice in ice-cold PBS and once in washing buffer [10 mM HEPES, pH 7.3, 110 mM KOAc, 2 mM Mg(OAc)2, 2 mM DTT]. They were then homogenized with hypotonic lysis buffer (5 mM HEPES pH 7.3, 10 mM KOAc, 2 mM Mg(OAc)2, 2 mM DTT, 20 μM cytochalasin B, 1 mM PMSF, 1 μg/mL each of leupeptin, pepstatin, and aprotinin). The supernatants were sequentially centrifuged at 1,500 g for 15 min, 15,000 g for 20 min, and 100,000 g for 1 h, dialyzed against transport buffer (TB; 20 mM HEPES, pH 7.3, 110 mM KOAc, 2 mM Mg(OAc)2, 5 mM NaOAc, 1 mM EGTA, 2 mM DTT, and 1 μg/mL each of leupeptin, pepstatin, and aprotinin), and frozen in aliquots in liquid nitrogen before storage at −80° C.

For the assays, HeLa cells were washed in TB and permeabilized for 5 min on ice in TB containing 40 μg/mL digitonin. The cells were rinsed for 5-10 min with several changes of TB, and the excess buffer was removed. The cells were incubated with transport mixture for 1 h at 22° C. The transport mixture contained HeLa cell cytosol at a final concentration of 2 mg/mL, which was preincubated for 30 min at room temperature with an ATP-regenerating system (1 mM ATP, 5 mM creatine phosphate, 20 U/mL creatine phosphokinase, 0.5 mM GTP) either with or without 10 μM OA, and 30 μg/mL of each substrate. The cells were fixed with 3.7% formaldehyde for 10 min and immediately examined by fluorescent microscope.

### Example 1.6 DNA Constructs and Mutagenesis

The gene encoding human HMGB1 was cloned upstream of GFP in pEGFP-N1 (BD Biosciences Clontech), and the construct was named pHMGB1-EGFP-N1. For the recombinant HMGB1-GFP protein, a SacI/NotI fragment from pHMGB1-EGFP-N1 was subcloned into pET-28a (Novagen). Six-His-tagged HMGB1-EGFP, GST-EGFP, and EGFP proteins were produced in E. coli BL21(DE3) pLysE (Novagen). The cells transformed with each construct were grown in LB medium containing kanamycin (15 μg/mL) and chloramphenicol (34 μg/mL) to an OD₆₀₀ of 0.4-0.5 at 37° C., cooled to 25° C., induced with 0.1 mM isopropyl-β-D-thiogalactopyranoside (IPTG), and grown overnight at 25° C. The cells were lysed by sonication, and the clear lysate was loaded onto a Ni²⁺-NTA column. The bound protein was washed with 50 mM NaH₂PO₄, 300 mM NaCl, and 20 mM imidazole, pH 8, and was eluted in the same buffer supplemented with 200 mM imidazole. All proteins were dialyzed into TB and stored at −80° C.

Site-directed mutations of HMGB1 were generated from pHMGB1-EGFP-N1 as a template using the QuickChange™ Site-directed Mutagenesis Kit (Stratagene). Human KAPs were cloned into BamHI/XhoI (KAPs-α1, α2, α4, α6), EcoRI/XhoI (KAPs-α3, α5) or BamHI/Not I (KAP-β1) sites of pGEX-4T-1 (Pharmacia) to produce GST-fusion proteins. KAP-α1 (accession: AAC60648), α2 (AAA65700), α3 (AAH17355), α4 (AAC25605), α5 (AAH47409), α6 (AAC15233), and β1 (AAH03572) were prepared from PCR amplifications of oligo(dT)-selected HeLa cell-derived cDNA. The primers were as follows:

KAP-α1

(SEQ ID NO: 1)

Forward
5′-CGCGGATCCATGACCACCCCAGGAAAAGAGAAC-3′,

(SEQ ID NO: 2)

reverse
5′-CCGCTCGAGAAGCTGGAAACCTTCCATAGGAGC-3′;
KAP-α2

(SEQ ID NO: 3)

Forward
5′-CGCGGATCCATGTCCACCAACGAGAATGCTAATAC-3′,

(SEQ ID NO: 4)

Reverse
5′-CCGCTCGAGAAAGTTAAAGGTCCCAGGAGCCCCAT-3′;
KAP-α3

(SEQ ID NO: 5)

forward
5′-CCGGAATTCATGGCCGAGAACCCCAGCTTGGAG-3′,

(SEQ ID NO: 6)

reverse
5′-CCGCTCGAGGGATCCCTCGAGAAACTGGAACCCTTCTGTT
GGTACA-3′
KAP-α4

(SEQ ID NO: 7)

forward
5′-CGCGGATCCATGGCGGACAACGAGAAACTGGAC-3′,

(SEQ ID NO: 8)

reverse
5′-CCGCTCGAGAAACTGGAACCCTTCTGTTGGTACA-3′;
KAP-α5

(SEQ ID NO: 9)

forward
5′-CCGGAATTCATGGATGCCATGGCTAGTCCAGGG-3′,

(SEQ ID NO: 10)

reverse
5′-CCGCTCGAGAAGTTGAAATCCATCCATTGGTGCTTC-3′;
KAP-α6

(SEQ ID NO: 11)

Forward
5′-CGCGGATCCATGGAGACCATGGCGAGCCCAGGG-3′,

(SEQ ID NO: 12)

reverse
5′-CCGCTCGAGTAGCTGGAAGCCCTCCATGGGGGCC-3′;
KAP-β1

(SEQ ID NO: 13)

Forward
5′-CGCGGATCCATGGAGCTGATCACCATTCTCGAGAAGACC-3′,

(SEQ ID NO: 14)

reverse
5′-ATAAGAATGCGGCCGCAGCTTGGTTGTTGACTTTGGTCAGTT
CTTTTG-3′.

The nucleotide sequences of restriction enzyme sites are underlined. The GST-KAP fusion proteins were produced in E. coli BL21. Cells were harvested and disrupted by sonication in lysis buffer with 1% Triton X-100, 10% glycerol, 1 mM EDTA, 1 mM DTT, and a protease inhibitor mix (1 μg/mL of leupeptin, pepstatin, and aprotinin and 1 mM PMSF) (Sigma) in PBS. After centrifugation, the supernatants were incubated with glutathione-Sepharose at 4° C. Bound proteins were eluted by incubation at room temperature for 30 min with 10 mM reduced glutathione. SDS-PAGE analysis of each eluted GST-KAP protein revealed a major protein band with the predicted molecular size. For the transfection study, Flag-tagged KAP-α1 was cloned into pCMV-Tag2 (Stratagene). All constructs were confirmed by DNA sequencing (Applied Biosystems). Fugene6 (Roche) was used for the transfection study.

### Example 1.7 Interaction Between KAP Protein and HMGB1 Mutant Proteins

Two μg of each GST-KAP protein was coupled to glutathione sepharose 4B beads and incubated with 500 μg of WCL from RAW 264.7 cells as a HMGB1 source at 4° C. overnight. WCLs were obtained after incubating cells in lysis buffer (50 mM HEPES pH 7.4, 150 mM NaCl, 1.5 mM MgCl2, 10% glycerol, 1% NP40, 1 mM EDTA, 50 mM NaF, 1 mM sodium-orthovanadate, 1 mM DTT, 1 mM PMSF, 10 μg/mL aprotinin, 10 μg/mL leupeptin, 10 μg/mL pepstatin) for 30 minutes on ice. Extracts were clarified by centrifugation at 20,000 g for 15 min at 4° C. GST complexes were washed and separated by 12% SDS-PAGE. The blots were probed with anti-HMGB1 and the signals were revealed by ECL detection as described above.

To observe the binding of KAP protein to each mutant HMGB1 in the cells, Flag-tagged KAP-α1 and each mutant HMGB1-GFP plasmid were co-transfected into RAW 264.7 cells. Cell homogenates of transfected RAW 264.7 cells were harvested and incubated with mouse anti-Flag (M2, Sigma) and mouse anti-GFP (Santa Cruz) at 4° C. overnight. Immune complexes were collected and the membranes were blotted with anti-Flag and anti-GFP, respectively. The reciprocal experiment was also performed. GST was used as a negative control. And to test the direct binding of HMGB1 to KAP-α1, GST pull-down assay was performed as described above.

Recombinant protein of HMGB1 was incubated with GST-KAP-α1 (10 μg) which was coupled to glutathione sepharose 4B beads. For this study, 6×His-tagged wild type HMGB1 and boxes A (aa 1-87) and B (aa 88-162) of HMGB1 were cloned into pRSETB (Invitrogen) and purified proteins were included in this test. After separating on the gel, the membrane was probed with anti-His and reprobed with anti-GST.

### Example 1.8 Metabolic Labeling

RAW 264.7 cells were cultured in phosphate starved condition of phosphate-free DMEM containing 10% dialyzed FBS (Gibco BRL) for 4 h and further incubated for 4 h by adding 600 μCi of [³²P]orthophosphate (Amersham Pharmacia Biotech) per mL to each dish. After 4 h, the cells were stimulated with 100 nM OA for 2 h, 100 ng/mL of LPS and 20 ng/mL of TNF-α for 2 and 8 h. The labeling was terminated by removing the culture medium followed by two immediate washes of the cells with ice-cold PBS. The cells were harvested by scrapping in 0.8 mL of lysis buffer (50 mM Tris, pH 7.5, 150 mM NaCl, 2 mM EDTA, 1% NP-40, 0.5% Sodium deoxycholate, 0.1% SDS, 50 mM NaF, 10 mM Sodium pyrophosphate, 25 mM β-glycerophosphate, 1 mM Sodium orthovanadate, 1 mM DTT, 10 μg/mL aprotinin, 10 μg/mL leupeptin, 5 μg/mL pepstatin, and 0.5 mM PMSF) and centrifuged at 21,000×g for 20 min at 4° C. The concentration of total soluble proteins in the supernatant was quantified using the Bradford reagent (Bio-Rad). Pre-cleared lysates were incubated with 2 μg/mL of rabbit anti-HMGB1 from two different companies of BD PharMingen and Upstate Biotechnology for 2 h at 4° C. Following the addition of protein G-Sepharose the tubes were rocked for an additional 1 h and beads were washed ten times with lysis buffer without SDS. Proteins were eluted in Laemmli sample buffer and separated. The gels were dried and the radioactivity was analyzed by autoradiography.

### Example 1.9 In Vitro Protein Kinase Assay of HMGB1

To investigate the enzyme that is involved in HMGB1 phosphorylation, in vitro protein kinase assay was performed. Six His-tagged wild type (WT) HMGB1 and HMGB1 NLS1/2A, both of which were not tagged with GFP, were purified from E. coli. HMGB1 NLS1/2A not tagged with GFP was cloned from the HMGB1 NLS1/2A. For the protein kinase C (PKC) reaction, 2 μg of each protein was incubated with 50 ng of PKC protein (Upstate) and 5 μCi [γ-³²P]ATP (Amersham Pharmacia Biotech) in a total volume of 50 μl buffer, which composed of 20 mM MOPS (pH 7.2), 25 mM β-glycerophosphate, 1 mM sodium orthovanadate, 1 mM DTT, 1 mM CaCl₂, lipid activator (10 μM PMA, 0.28 mg/ml phosphatidylserine, 0.3% Triton X-100 mixed micelle suspension) and 50 μM ATP. And for casein kinase 11 (CK II) reaction, 500 U of CK II (New England BioLabs) was used in the buffer, which composed of 20 mM Tris-HCl (pH 7.5), 50 mM KCl, 10 mM MgCl₂, 50 μM ATP. For the cdc2 reaction, 20 U of Cdc2 kinase (New England BioLabs) was used in the buffer, which composed of 20 mM Tris-HCl (pH 7.5), 10 mM MgCl₂, 1 mM EGTA, 2 mM DTT, 50 μM ATP. All the reactions were performed at 30° C. for 40 min. Each reaction was terminated by adding 5× sample buffer. The samples were separated with 12% SDS-PAGE and autoradiography was performed after drying.

### Example 1.10 Inhibition of HMGB1 Secretion by Protein Kinase C Inhibitor

To observe the inhibition of HMGB1 secretion by PKC inhibitor, RAW 264.7 cells were treated with 200 ng/mL of LPS alone or combined with Gö 6983 (Calbiochem), a conventional PKC (α, β, and γ) inhibitor including PKCδ and PKCζ, at the indicated concentrations for 16 h. Other inhibitors of SB203580 (p38 inhibitor, Calbiochem), PD098059 (ERK1/2 inhibitor, Calbiochem), Bay 11-7082 (NF-κB inhibitor, Calbiochem) were included. The supernatants were harvested and separated with 12% SDS-PAGE and Western blot was performed to observe the level of HMGB1.

### Example 2

### HMGB1 Serine Residues are Phosphorylated by TNF-α and OA Treatments

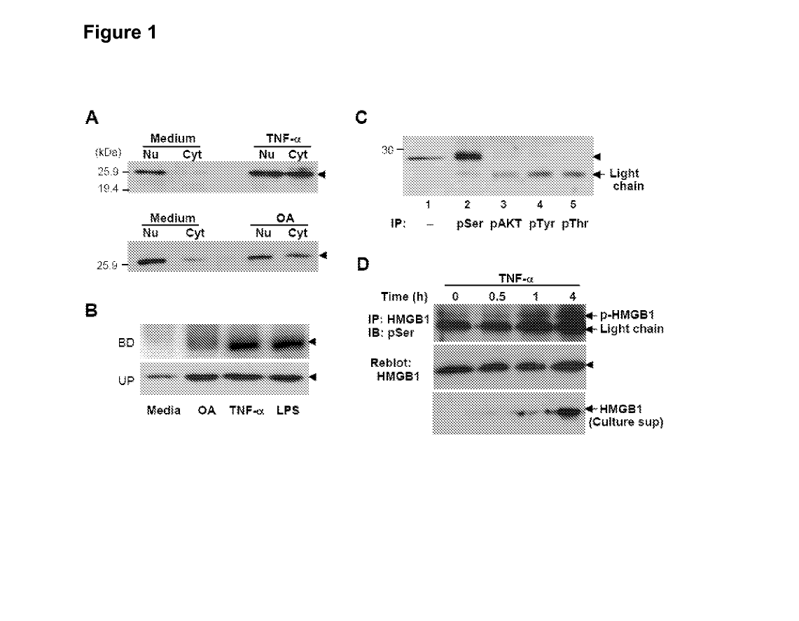

To investigate whether HMGB1 is phosphorylated and how its phosphorylation influences its nuclear transport, RAW 264.7 cells were treated with OA, a type 1/2A protein phosphatase inhibitor, to induce forced phosphorylation of HMGB1. OA was used at a low concentration of 100 nM for 8 h or less to minimize the nuclear leakage of HMGB1 and block entry into the cell cycle. Treatment with TNF-α as a positive control cytokine resulted in the translocation of nuclear HMGB1 to the cytoplasm (FIG. 1A, upper panel). Treatment of cells with OA also resulted in increased levels of HMGB1 in the cytoplasm (FIG. 1A, lower panel) similar to that seen with TNF-α-treated cells.

Next, to demonstrate the direct evidence of HMGB1 phosphorylation, RAW 264.7 cells were labelled with [³²P]orthophosphate and stimulated with 100 nM OA, 20 ng/mL TNF-α and 100 ng/mL LPS for the indicated length of time, and the cell lysates were immunoprecipitated with anti-HMGB1 for autoradiography. We used rabbit anti-HMGB1 from two different vendors for confirmation. HMGB1 was phosphorylated by OA, and by TNF-α and LPS treatments. Phosphorylation was increased by the increment of treatment time from 2 h to 8 h (FIG. 1B).

To determine which amino acid residue of HMGB1 is phosphorylated, RAW 264.7 cells were treated with 20 ng/mL TNF-α for 16 h. WCLs from treated cells were immunoprecipitated with anti-pSer, anti-pTyr and anti-pThr antibodies, separated, and immunoblotted with anti-HMGB1. Only serine residues of HMGB1 were phosphorylated by TNF-α treatment (FIG. 1C). Next, RAW 264.7 cells were treated with TNF-α for the indicated length of time, and the culture supernatants were harvested and concentrated to observe HMGB1 secretion. WCLs were immunoprecipitated with anti-HMGB1 and then immunoblotted with anti-pSer. The levels of HMGB1 were nearly unchanged within whole cells but were increased in the culture supernatants (FIG. 1D, middle and lower panels), confirming the time-dependent secretion of HMGB1. The level of phosphorylated (p-) HMGB1 was also increased by TNF-α in a time-dependent manner (FIG. 1D, upper panel).

### Example 3

### Phosphorylated HMGB1 is Relocated Towards Secretion

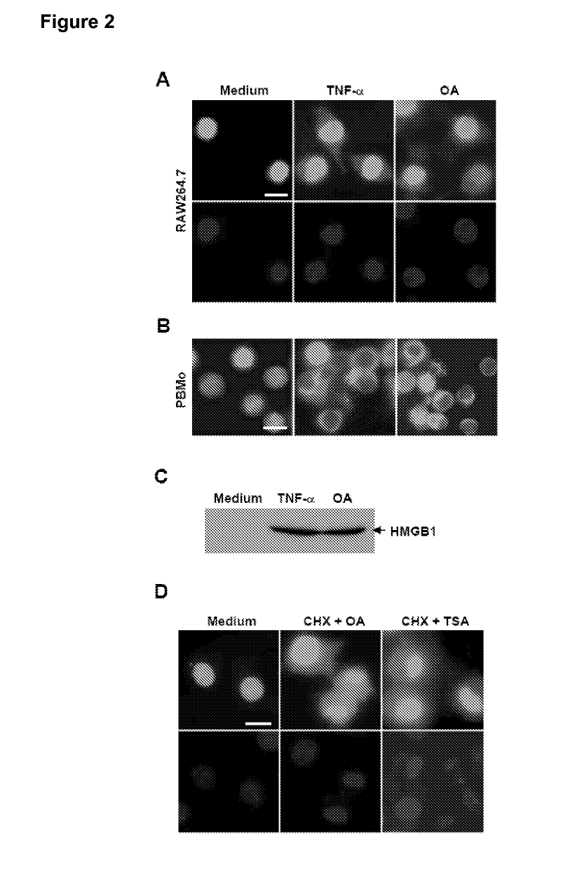

To further examine the effect of phosphorylation on the relocation of HMGB1, RAW 264.7 cells were treated with OA, and indirect immunofluorescent staining was performed. HMGB1 was mostly observed in the nuclei of unstimulated RAW 264.7 cells (FIG. 2A). When the cells were treated with OA for 8 h, HMGB1 was observed in both the nucleus and the cytoplasm, which was similar to that seen in TNF-α-treated cells (FIG. 2A). Relocation of HMGB1 after OA treatment was also clearly observed in freshly isolated human PBMo cells (FIG. 2B), confirming the phosphorylation effect of HMGB1. The same culture supernatants of human PBMo cells were harvested to observe the secreted HMGB1. HMGB1 was detected in the culture supernatants of PBMo cells after OA treatment (FIG. 2C), suggesting the relation of HMGB1 secretion to its phosphorylation.

To exclude the possibility of HMGB1 presence in the cytoplasm due to new protein synthesis, HMGB1 relocation was directly observed using HMGB-GFP plasmid after treatment with CHX, an inhibitor of any new protein synthesis. RAW 264.7 cells were transfected with wild-type HMGB1-GFP plasmid, incubated for 24 h, and then treated with 2 μg/mL CHX. One h after CHX treatment, OA was added for 4 h in the presence of CHX. As shown in FIG. 2D, HMGB1-GFP protein, which was mostly observed in the nuclei of the cells 24 h after transfection, was relocated to the cytoplasm after OA treatment in the presence of CHX. This result suggests that HMGB1 observed in the cytoplasm after phosphorylation is not due to new protein synthesis but due to relocation of existing proteins inside the nucleus. We treated TSA, a histone deacetylase inhibitor for 2 h, as a positive control because the hyperacetylated HMGB1 is relocated from the nucleus to the cytoplasm.

### Example 4

### Phosphorylated HMGB1 in the Cytoplasm does not Enter the Nucleus (Nuclear Import Assay)

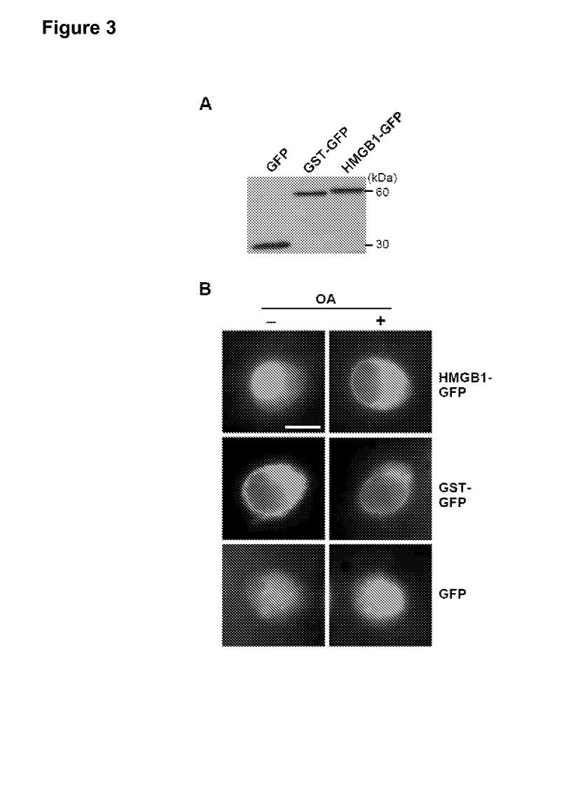

HMGB1 can transverse the nuclear membrane in both directions, however, HMGB1 molecules are predominantly in the nucleus in an unstimulated state, indicating that import is much more effective than export. To further demonstrate whether phosphorylation influences nuclear import of HMGB1, a nuclear import assay was performed using a digitonin-permeabilized HeLa cell-free transport system (Adam, S. A., R. S. Marr, and L. Gerace. J Cell Biol 111:807-816 (1990)) Digitonin-permeabilized cells have perforated plasma membranes, which release cytosolic components from cells while the nuclear envelope and other major organelle membranes remain intact. As a source of exogenous HMGB1 protein, recombinant HMGB1-GFP protein was purified from E. Coli (FIG. 3A). Glutathione-5-transferase (GST)-GFP was prepared as a control protein.

HMGB1-GFP was observed in the nuclei of digitonin-treated HeLa cells when the cells were incubated for 1 h with the transport mixture that contained HMGB1-GFP but not OA, suggesting that HMGB1-GFP entered the nucleus by default way (FIG. 3B, upper left). When the cells were incubated with the HMGB1-GFP-containing transport mixture that included OA, HMGB1-GFP remained in the cytoplasm (FIG. 3B, upper right). The GST-GFP protein did not enter the nucleus, regardless of whether the transport mixture was treated with OA or not (FIG. 3B, middle). GST has no NLS and thus was located in the cytoplasm regardless of its phosphorylation in the presence of an ATP-regenerating system. Finally, unfused GFP was distributed throughout the cells (FIG. 3B, lower), which is a well-known observation. These results show that the phosphorylation of HMGB1 occurring in the cytoplasm prevented its nuclear import and plays a critical role in localizing HMGB1 to the cytoplasm. HMGB1 has two NLSs for nuclear import. Therefore, the phosphorylation of HMGB1 at a region close to both NLSs may possibly play an important role in the controlled shuttling mechanism of HMGB1.

### Example 5

### HMGB1 Binds to KAP-α1 and Phosphorylation of HMGB1 Decreases its Binding to KAP-α1

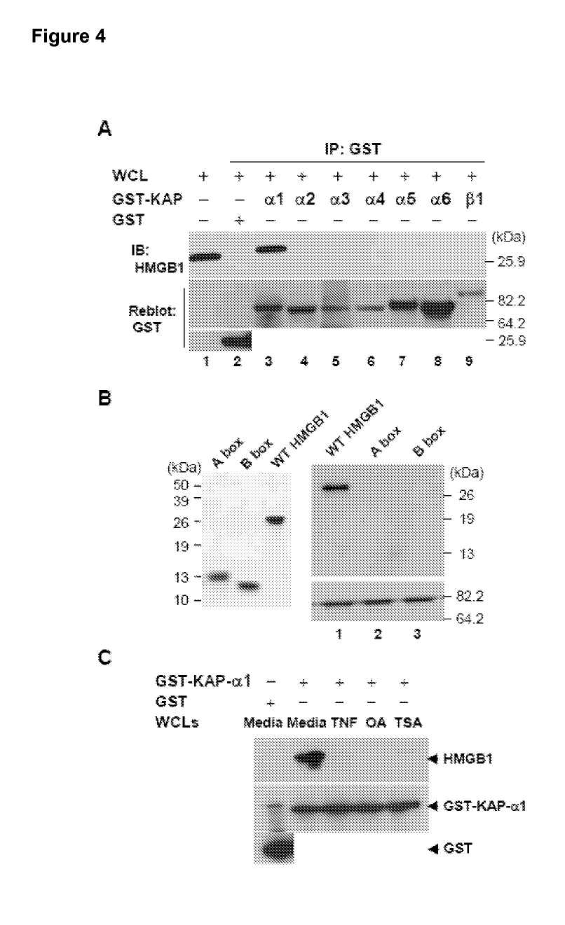

To investigate whether phosphorylation prevents HMGB1 from interacting with the nuclear import protein, it was determined which KAP protein is involved in binding with HMGB1 as its cargo protein and then observed the interaction of p-HMGB1 with the KAP protein. The KAP family proteins act as shuttling receptors and specifically bind the NLS motifs of cargo proteins to facilitate their nuclear import (Rendon-Mitchell, B. et al, J Immunol 170:3890-3897 (2003)]. For this study, GST-KAP fusion proteins of α1, α2, α3, α4, α5, α6, β1 were produced in E. coli. GST-KAP-β1 was included because some proteins directly bind to KAP-β1 for their nuclear transport. For an in vitro protein-protein interaction study, WCLs of unstimulated RAW 264.7 cells, a source of unphosphorylated HMGB1, were incubated with each GST-KAP fusion protein, which was bound to glutathione-Sepharose beads. KAP-α1 was identified as the carrier protein for HMGB1 (FIG. 4A).

Direct binding of recombinant HMGB1 protein to KAP-α1 was tested to exclude the possibility that other HMGB1-interacting proteins present in cell lysates could have a role in binding. Purified six His-tagged wild type HMGB1 protein and boxes A (aa 1-87) and B (aa 88-162) proteins, which were identified at expected size by Coomassie blue staining (FIG. 4B, left), were purified from E. coli BL21. Same molar amounts of all wild and truncated forms of HMGB1 were added to GST-KAP-α1, and GST pull-down assay was performed. Only wild type HMGB1 was found to bind GST-KAP-α1 (FIG. 4B, right), showing the direct binding of HMGB1 to KAP-α1 without any other interacting proteins. Box A and B proteins, which include NLS1 (aa 28-44) and no NLS respectively, showed no binding.

We next tested the binding of p-HMGB1 to KAP-α1 protein. When the binding of KAP-α1 to OA-treated RAW 264.7 cell lysate was tested, the interaction was not observed while the binding to medium-treated RAW 264.7 cell lysate was clearly seen (FIG. 4C). This result demonstrates that phosphorylation of HMGB1 is an important modification that decreases its nuclear import by reducing the binding to KAP-α1. Acetylated HMGB1 from TSA-treated RAW 264.7 cell lysate showed no binding to KAP-α1, implying that re-entry of acetylated HMGB1 to the nucleus is blocked because of no binding to KAP-α1.

### Example 6

### The Effect of Phosphorylation of Both NLS Regions of HMGB1 on the Binding to KAP-α1

HMGB1 was phosphorylated at serine residues (FIG. 1C), thus it is proposed that serine phosphorylation close to either or both NLSs is crucial for its relocation. There are 11 serines throughout the HMGB1. Among them, five serines are at 35, 39, 42, 46 within NLS1 and at 181 within NLS2. The NetPhos 2.0 program (www.cbs.dtu.dk/services/NetPhos/) predicts six serines as the possible phosphorylation sites. They are five serines within NLS1 and NLS2 described above and one more serine at 53 close to NLS1.

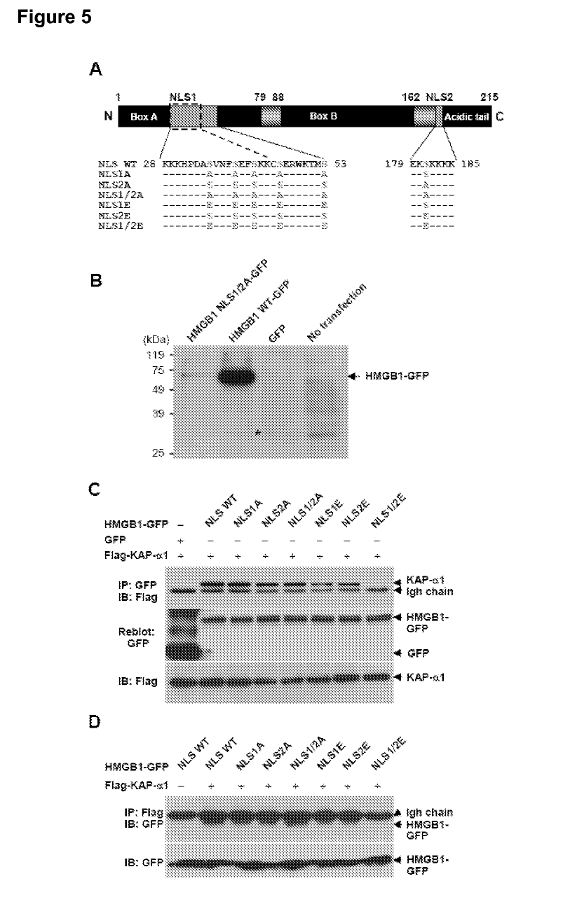

To observe the effect of phosphorylation in both NLS regions of HMGB1 on the binding to KAP-α1 and on the subcellular localization of HMGB1 in the transfected cells, a number of site-directed mutations in six serines of NLS1 and NLS2 were generated using a HMGB1-GFP fusion construct plasmid (FIG. 5A). Serines 35, 39, 42, 46, 53 within or close to NLS1 and serine 181 at NLS2 were alternatively or totally mutated into alanine or glutamic acid. Substitution with alanine and glutamic acid simulated unphosphorylated and a phosphorylated states, respectively. RAW 264.7 cells were co-transfected with a Flag-tagged KAP-α1 plasmid and each mutant HMGB1-GFP plasmid, and immunoprecipitates using anti-GFP (FIG. 5B) or anti-Flag (FIG. 5C) were analyzed with anti-Flag for KAP-α1 or anti-GFP for HMGB1. As shown in FIGS. 5B and 5C, the interactions of HMGB1 NLS1A, NLS2A, and NLS1/2A with KAP-α1 were similar or slightly decreased compared to HMGB1 NLS WT. Those of HMGB1 NLS1 E and NLS2E, which mimicked the phosphorylation in either NLS regions, were significantly decreased to about 50% of wild-type. They were predominantly observed in the nucleus, possibly suggesting a slow entrance to the nucleus. However, HMGB1 NLS1/2E showed no binding to KAP-α1. These results suggest that phosphorylation level at either or both NLSs of HMGB1 differentially reduces the binding to KAP-α1 and has a significant impact on the nuclear import of HMGB1.

### Example 7

### Phosphorylation of Both NLS Regions of HMGB1 is Required for its Relocation to the Cytoplasm

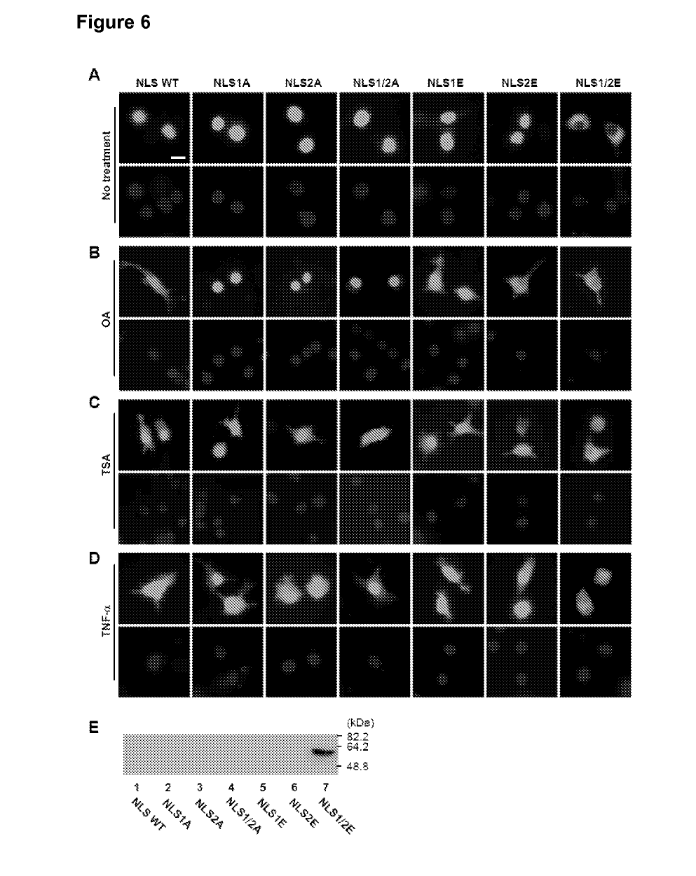

The movement of HMGB1 by the state of HMGB1 phosphorylation at either or both NLSs was investigated. RAW 264.7 cells were transfected with each mutant HMGB1-GFP plasmid and cultured for 24 h without any stimulation. Then the fluorescent images were observed. The mutant fusion proteins from the HMGB1 NLS1A, NLS2A, NLS1/2A, NLS1E, and NLS2E constructs, which showed the interaction with KAP-α1 at least about 50% compared to wild-type, were localized to the nuclei 24 h after transfection (FIG. 6A). HMGB1 NLS1/2E, however, mimicking the phosphorylation at both NLSs, was located in the cytoplasm. When the same culture supernatants were harvested to observe the secreted HMGB1, HMGB1-GFP protein was detected only in HMGB1 NLS1/2E-transfected cells (FIG. 6E). These data strongly suggest that the concomitant change to the phosphorylated state at both NLSs is important for its cytoplasmic localization and subsequent secretion. When the transfection study was carried out using the nonmyeloid HeLa cell line, similar results were also obtained (data not shown).

Next, RAW 264.7 cells were treated with OA 24 h after each transfection to further investigate the effect of change of phosphorylation state on HMGB1 relocation. HMGB1 NLS WT, NLS1E, and NLS2E were relocalized to the cytoplasm afterOAtreatmentwhile HMGB1 NLS1A, NLS2A, and NLS1/2A were unaffected (FIG. 6B). HMGB1 NLS1A, NLS2A and NLS1/2A remained in un-phosphorylated states at either or both NLS regions even after OA treatment. Hence, HMGB1 relocation to the cytoplasm occurred by phosphorylation of both NLS regions. When RAW 264.7 cells were treated with TSA, which induce forced acetylation of HMGB1 regardless of serine phosphorylation, the wild-type and all mutant HMGB1-GFPs showed cytoplasmic relocation (FIG. 6C). The same results were observed in the TNF-α-treated cells (FIG. 6D). Proinflammatory signaling pathways by TNF-a has the impact on the enzymes responsible for acetylation/deacetylation and also phosphorylation in our data. These results show that phosphorylation of both NLS regions of HMGB1 is involved in the cytoplasmic relocation in addition to acetylation and its eventual secretion.

### Example 8

### Protein Kinase C Phosphorylates HMGB1

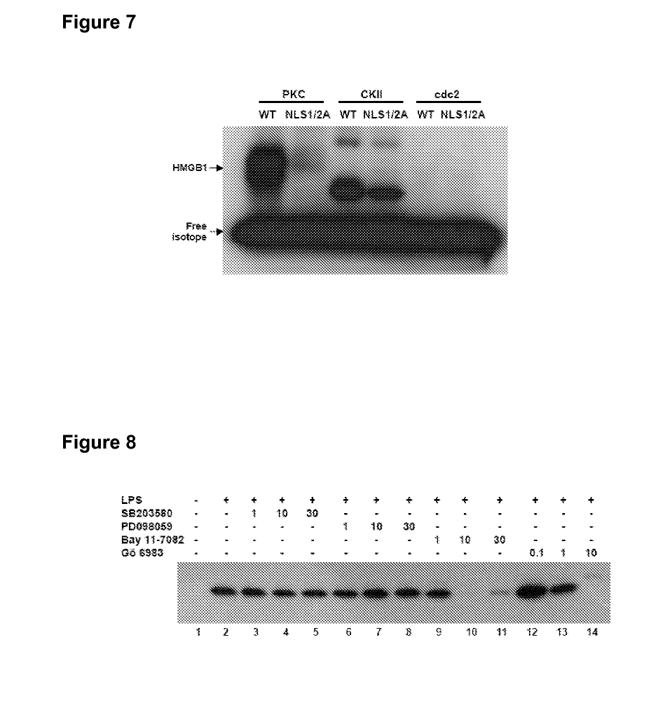

To investigate the enzyme that is involved in HMGB1 phosphorylation, in vitro protein kinase assay was performed. For this assay, PKC, CK II and cdc2 protein kinases were tested because these enzymes were known to phosphorylate HMG-I of HMG family protein (Xiao D M, et al. J Neurochem 74:392-399 (2000), Nissen M S, et al. J Biol Chem 266:19945-19952 (1991), Reeves R, et al. Proc Natl Acad Sci USA 88:1671-1675 (1991), Palvimo J and Linnala-Kankkunen A, FEBS Lett 257:101-104 (1989)). As shown in FIG. 7, PKC phosphorylated WT HMGB1 but CK II and cdc2 showed no phosphorylation of HMGB1, indicating that PKC is the main protein kinase of HMGB1 protein. HMGB1 NLS1/2A protein, however, showed little or no phosphorylation compared to the WT HMGB1, suggesting again that six serines in NLS 1 and NLS2 are the main phosphorylation sites.

### Example 9

### Effect of Protein Kinase C Inhibitor in the Secretion of HMGB1

The secretion of HMGB1 was evaluated to determine whether it is inhibited by PKC inhibitor to confirm the involvement of PKC in HMGB1 phosphorylation. RAW 264.7 cells were treated with 200 ng/mL of LPS alone or combined with Go 6983 at the indicated concentrations for 16 h. The levels of HMGB1 in the culture supernatants were decreased to undetectable level by increasing the concentration of Go 6983 from 0.1 to 10 μM (FIG. 8). However, SB203580 and PD098059, which are p38 and ERK1/2 inhibitors respectively, showed no change of HMGB1 secretion. When the cells were treated with Bay 11-7082, an NF-κB inhibitor, the secretion of HMGB1 was decreased in a concentration-dependent manner, possibly because the activity of histone acetyl transferase (HAT) was inhibited by NF-κB inhibitor (Bonaldi, T. et al. Embo J 22:5551-5560 (2003)).

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A pharmaceutical composition comprising an antagonist of HMGB1 phosphorylation.

2. A pharmaceutical composition according to claim 1, wherein the antagonist is a protein kinase inhibitor or a protein phosphatase activator.

3. A pharmaceutical composition according to claim 2, wherein the protein kinase inhibitor is Protein Kinase C inhibitor.

4. A method of identifying an agent that regulates the phosphorylation of HMGB1, comprising the steps (a) determining the level of the phosphorylated HMGB1 in the presence and absence of said agent; (b) comparing the level of the protein determined in step (a); and (c) identifying said agent as a regulator by the differences in the phosphorylation of HMGB1 activity in the presence or absence of said compound.

5. A method for treating a condition associated with activation of the inflammatory cytokine cascade comprising administering an effective amount of an antagonist of HMGB1 phosphorylation.

6. The method according to claim 5, wherein the condition is sepsis or a related condition.

7. The method according to claim 5, wherein the antagonist is a protein kinase inhibitor or a protein phosphatase activator.

8. The method according to claim 7, wherein the protein kinase inhibitor is a protein kinase C inhibitor.

9. A method for diagnosis and or prognosis of the conditions associated with the activation of the inflammatory cascade comprising measuring the concentration of phosphorylated protein HMGB1 in a sample, and comparing that concentration to a standard for phosphorylated protein representative of a normal concentration range of phosphorylated HMGB 1 in a like sample, whereby higher levels of phosphorylated HMGB1 are indicative of the disease.

10. The method of claim 9, wherein the sample is a serum sample.


SEQUENCE LISTING (14 sequences)

SEQ ID NO: 1 - DNA, 33 bp, Artificial Sequence
  the forward primer of KAP-a1
cgcggatccatgaccaccccaggaaaagagaac

SEQ ID NO: 2 - DNA, 33 bp, Artificial Sequence
  the reverse primer of KAP-a1
ccgctcgagaagctggaaaccttccataggagc

SEQ ID NO: 3 - DNA, 35 bp, Artificial Sequence
  the forward primer of KAP-a2
cgcggatccatgtccaccaacgagaatgctaatac

SEQ ID NO: 4 - DNA, 35 bp, Artificial Sequence
  the reverse primer of KAP-a2
ccgctcgagaaagttaaaggtcccaggagccccat

SEQ ID NO: 5 - DNA, 33 bp, Artificial Sequence
  the forward primer of KAP-a3
ccggaattcatggccgagaaccccagcttggag

SEQ ID NO: 6 - DNA, 46 bp, Artificial Sequence
  the reverse primer of KAP-a3
ccgctcgagggatccctcgagaaactggaacccttctgttggtaca

SEQ ID NO: 7 - DNA, 33 bp, Artificial Sequence
  the forward primer of KAP-a4
cgcggatccatggcggacaacgagaaactggac

SEQ ID NO: 8 - DNA, 34 bp, Artificial Sequence
  the reverse primer of KAP-a4
ccgctcgagaaactggaacccttctgttggtaca

SEQ ID NO: 9 - DNA, 33 bp, Artificial Sequence
  the forward primer of KAP-a5
ccggaattcatggatgccatggctagtccaggg

SEQ ID NO: 10 - DNA, 36 bp, Artificial Sequence
  the reverse primer of KAP-a5
ccgctcgagaagttgaaatccatccattggtgcttc

SEQ ID NO: 11 - DNA, 33 bp, Artificial Sequence
  the forward primer of KAP-a6
cgcggatccatggagaccatggcgagcccaggg

SEQ ID NO: 12 - DNA, 34 bp, Artificial Sequence
  the reverse primer of KAP-a6
ccgctcgagtagctggaagccctccatgggggcc

SEQ ID NO: 13 - DNA, 39 bp, Artificial Sequence
  the forward primer of KAP-b1
cgcggatccatggagctgatcaccattctcgagaagacc

SEQ ID NO: 14 - DNA, 48 bp, Artificial Sequence
  the reverse primer of KAP-b1
ataagaatgcggccgcagcttggttgttgactttggtcagttcttttg